(19) United States
(12) Patent Application Publication
(10) Pub. No.: US 2008/0269590 A1
(43) Pub. Date: Oct. 30, 2008

(54) MEDICAL INSTRUMENT FOR PERFORMING A MEDICAL INTERVENTION

(76) Inventor: Matthias Wedel, Nurnberg (DE)

Correspondence Address: BRINKS HOFER GILSON & LIONE, P.O. BOX 10395, CHICAGO, IL, 60610, US

(21) Appl. No.: 12/105,739

(22) Filed: Apr. 18, 2008

(30) Foreign Application Priority Data
Apr. 24, 2007 (DE) DE102007019333.7

(51) Int. Cl. A61B 5/00; A61B 17/00

(52) U.S. Cl. 600/407; 606/1

(57) ABSTRACT

A medical instrument for performing a medical intervention on a body region is provided. The medical instrument includes a sensor for recording tissue parameters of a body region to be treated using measuring techniques.

This application claims the benefit of DE 10 2007 019 333.7, filed Apr. 24, 2007, which is hereby incorporated by reference.

### BACKGROUND

The present embodiments relate to a medical instrument for performing a medical intervention.

A medical instrument may be used for medical intervention. Medical instruments include scalpels, high frequency scalpels (HF knives), endoscopes, surgical lasers or other medical instruments used for the intervention. During a medical intervention, the medical instrument may destruct healthy body tissues. The destruction of healthy body tissues may impact the operation methods used for the intervention.

An operating physician visually determines whether the body tissue is healthy or diseased body tissue. The visual check is difficult to perform during a keyhole surgery, since small fields of view are available. The small fields of view make it difficult to see and survey the operating field. The identification of tissues may be hampered by contamination at the operating site.

DE 10 2005 022 120 A1 describes the use of a sensor and a catheter. The sensor may be used to position the catheter. At a free end of the catheter, the catheter includes a positioning indicator. Applying an external magnetic field causes an interaction. The interaction may be used to determine the position in a three-dimensional coordinate system.

EP 0 883 375 B1 discloses using a sensor or a plurality of sensors to determine the position of endoscopes. The sensors are located in the top part of the endoscope and transmit or receive magnetic fields, so that the position of the sensor and the endoscope in the body may be determined.

EP 0 872 211 A1 describes a device for identifying various tissue types to detect precancerous or cancerous activity (tissue). The probe includes an emitter for irradiating the tissue and a detector tip that receives the returned radiation, The probe may include an electrode and an electrical device for measuring the resulting electrical reaction. The optical or electrical signals may be compared with known tissue type signals, so that the examined tissue can be assigned to a specific type.

DE 20 2005 011 442 U1 discloses a device for non-invasive identification of tissue and/or of the state of the tissue. An optical device is used that is coupled with a recording device. The data captured by the recording device is compared in an evaluation device with comparative data of known tissues so that the tissue type and the state of the tissue can be determined.

### SUMMARY AND DESCRIPTION

The present embodiments may obviate one or more of the drawbacks or limitations inherent in the related art. For example, in one embodiment, a medical instrument improves identification of different body tissue types.

In one embodiment, a medical instrument for performing a medical intervention on a body region includes at least one sensor that records tissue parameters of a body region to be treated and/or operated on using measuring techniques. The recorded tissue parameters may be used to distinguish diseased tissue from healthy tissue. Identification of healthy and diseased tissue makes it possible to avoid an erroneous injury or removal of healthy tissue. The medical instrument is coupled with a sensor that may distinguish tissue types from one another.

The distinction of tissue types may be performed in various ways and is based on different features of the tissue types and/or of their direct environment. For example, different tissue types may be distinguished according to different optical properties and/or reflection properties when illuminated with visible, ultraviolet and/or infrared light. In another example, different tissue types may be distinguished according to different chemical, physical, physical-chemical, biomechanical and biochemical properties, such as differences in conductivity, in electrical potential, or in viscoelasticity of tissues. By making use of the Doppler effect, the self-movement of tissue, for example, of arteries or veinsmay be identified.

The distinguishing features may be identified and measured with an optical and/or electronic sensor. As a result of these measured values and on the basis of known standard values the analyzed tissue samples may be assigned to different tissue types. A distinction may be made between various tissue types (e.g. nerves, blood vessels, muscle, skin, fat, bone, malignant tissue, or other tissue). Tissue types and tissue changes may be identified through the use of corresponding markers. For example, infrared fluorescent dyes may be used to mark cancerous tissue.

The sensor for recording the treated body region may generate a measurement signal that is characteristic of a tissue type and may be coupled with a suitable data evaluation system.

In one embodiment, a currently analyzed tissue type may be shown or marked optically so that the operating doctor receives a sensor evaluation in addition to his own assessment. A distinction may be made between vulnerable tissues, non-vulnerable tissues, and desired tissues. Vulnerable tissues may include nerves or blood vessels. Non-vulnerable tissues may include muscle, skin, fat, or bone. Desired tissuemay include malignant tissue. The assignment of different tissues to a certain response type may be stored as part of a standard configuration. The assignment may be performed during operation planning and adjusted to the specific situation of the patient. An evaluation unit, which is assigned to the data evaluation system for evaluating the measurement signals, may include a signal processing unit that may subdivide the examined body tissue into vulnerable and non-vulnerable body tissue and/or to identify desired (classified) tissue types, such as malignant tissue.

The identified tissue class may be coupled (associated) to a specific action. The action may be, for example, the output of status reports. The evaluation unit may include an output unit for outputting optical and/or acoustic signals. Optical and/or acoustic warning signals may be generated. A textual or graphical reproduction may be displayed on the instrument or on a unit coupled to the instrument which uses measuring techniques and/or is computer-controlled. The information may be transmitted via further services and/or individuals, for example, using pagers, e-mail, or short message service (SMS).

In one embodiment, the evaluation unit is coupled to the medical instrument. The information described above may be used directly to control the medical instrument. For example, the power output and/or positioning configuration and/or focussing of a surgical laser used as a medical instrument may be controlled. The function of the instrument may be restricted or interrupted if healthy (non-vulnerable) tissue or nerves and/or blood vessels (vulnerable tissue) are identified, in order to prevent healthy tissue and/or the vulnerable areas from being wrongly removed and/or damaged.

If the specifically desired tissue (e.g., malignant tissue) is identified because of specific properties or the presence of corresponding markers, functions of the instrument may be activated so that desired tissue is removed. Further devices (peripheral devices) which are not themselves directly fitted (coupled) with a sensor, may be actuated on the basis of the available information. The peripheral devices may include, for example, suction devices or rinsing devices. For coupling purposes, the evaluation unit may be developed with at least one further processing device.

The sensor may have a transmitter and receiver. For example, an optical sensor may have a transmitter, which emits light at a certain wavelength onto the tissue to be examined, and a receiver, which detects the reflected and/or scattered light and generates a measurement signal therefrom. Other sensors may be used, for example, pH-value measurements may also take place. A sensor may detect and measure a plurality of parameters so that an even more precise identification of tissue becomes possible.

The sensor may be used with and/or fitted to medical instruments, such as endoscopic instruments, surgical instruments, medical robots and surgical lasers. The sensor may be used with material processing. For example, if the defective material displays different properties to the normal material. The differing properties may be detectable. For example, chemical and/or physical properties are possible. The different properties may be different conductivity, different light transmission, or different reflection of light at a certain wavelength.

A notification can be output that corresponds to the tissue type when a high frequency knife (with a sensor) approaches an identified tissue. If the identified tissue is an arterial vessel, a nerve cord, a bile duct or another vulnerable tissue that can on no account be injured, a warning notification may be output and the function of the device blocked. Alternatively, if the instrument or device was not yet in action, then the warning, coupled with the automatic deactivation, prevents activation of the device. The corresponding tissue is prevented from being injured. So the operating doctor would be warned and could, for example, substitute the operating tool and use a tool that would be less injurious to the tissue. If, for example, an endoscope included a sensor that could distinguish between blood and/or lymph, the identification of blood and/or lymph could be coupled directly with an action, namely rinsing of the operating field.

A medical instrument may be provided with a sensor for recording tissue parameters. The tissue identification, linked with the simultaneous coupling to a warning function and the further coupling in the form of deactivation signals or or the like, prevents vulnerable tissue (e.g., nerve bundles, bile ducts, or blood vessels) from being wrongly damaged during the operation. If such sensors are used in surgical robots, the robots may be controlled on the basis of further parameters. The use of sensors improves safety when applying lasers, for example, to remove diseased tissue.

Methods in which the sensor described above can be used together with a medical instrument, with tissue being identified by the sensor. The function of the treatment device and/or further peripheral devices can be influenced by the identification.

### BRIEF DESCRIPTION OF THE DRAWINGS

FIG. 1 shows one embodiment of a medical instrument that is fitted with a sensor for tissue identification.

### DETAILED DESCRIPTION

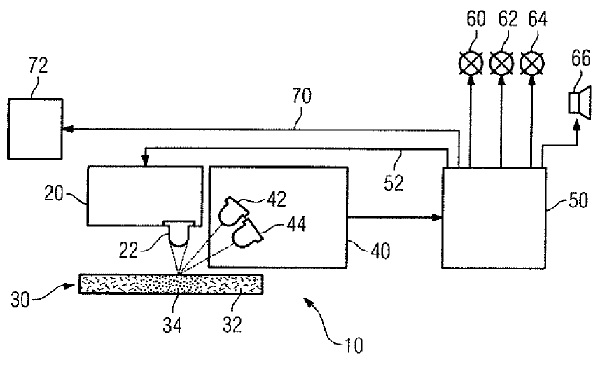

FIG. 1 shows an operating device 10. The operating device 10 includes a medical instrument 20, a sensor 40, and an evaluation unit 50. The medical instrument may be a scalpel, a laser device or other similar instrument. The sensor 40 may record parameters of the treated tissue 30. The evaluation unit 50 may output control signals and/or warning signals. The operating device 10 may include additional components.

During a medical intervention, a medical instrument 20 may be used to treat the tissue 30 of a patient. An operation may be carried out to remove diseased tissue 34. The diseased tissue 34 may be, for example, cancerous tissue that is completely removed, with as little healthy tissue 32 as possible being removed. Vulnerable tissues (e.g., nerve tracts, blood vessels, bile ducts) should not be damaged.

As shown in FIG. 1, the medical instrument 20 may include a laser device 22 that removes diseased tissue 34. The sensor 40 is assigned to the laser device 22. The sensor 40 may be, for example, an optical sensor in which a transmitter 42 emits light at a certain wavelength onto the tissue 30 to be treated. A receiver 44 records the illuminated area and the light reflected from the illuminated area. A distinction between nerve tissue, other tissue and malignant cancerous tissuemay be made on the basis of different optical properties of these tissue types. Fluorescent markers may be used. The flourescent markers may only adhere to and unambiguously mark cancerous cells.

The values measured by the sensor 40 are sent to the evaluation unit 50. The evaluation unit 50 evaluates the values by an integrated signal processing unit. For example, the values measured by the receiver 44 are compared with known data about tissue structures, The tissue of the area to be treated is classified according to the comparison. Electronic sensors may be used. Electronic sensors may measure the conductivity of the tissue to be treated and to classify the tissue accordingly on the basis of comparative data. Based on the classification performed, the evaluation unit 50 can actuate different signals. For example, visual warning signals 60, 62 and 64 and/or acoustic warning signals 66 may be output.

The evaluation unit 50 may trigger (generate) a signal 52 if diseased tissue 34 is identified. The signal 52 may cause the activation of a processing device 2. For example, the laser beam of the laser device 22 may be activated. A visual signal 60 may be output in parallel. The illumination of a green light signal may indicate to the doctor that diseased tissue 34 has been identified and that the treatment device 20 has been activated.

The evaluation unit 50 may generate a visual signal 62, if healthy, non-vulnerable tissue is identified. This visual signal 62 may activate a small red light or a red LED and signal to the operating person that this tissue should not be removed, but also that there is no danger of injury to nerve tracts, blood vessels. The treatment device 20 may be deactivated automatically.

The evaluation unit 50 may trigger (generate) a visual signal 64 if healthy, but vulnerable tissue (nerve tracts, blood vessels) is identified. This visual signal 64 may be generated by activating a small blue light or a blue LED and may signal to the operating person that this tissue should not be removed and also that there is a danger of injury to nerve tracts, blood vessels. The treatment device 20 may be deactivated automatically.

The color assignment for different visual warning signals described above may be different and is to be understood merely as an example. The doctor performing the treatment may also receive notification of the optical signals by alternative notifications, for example, in the form of visual projections using data glasses. The information is not overlooked, but does not cause the doctor performing the treatment to be obliged to observe a signal display, for which it might be necessary for him to turn his head. Distracting the doctor through the type of information transmission may be avoided.

The visual warning signals may be complemented or amplified by acoustic warning signals 66. Different melodies and/or lengths and/or pitches of sounds (acoustic signals 66) may be assigned to the different tissue types.

The different tissue types may shown textually on a display. For example, the sensor 40, the evaluation unit 50 and the visual and/or acoustic warning and/or action trigger may be integrated in a device, so that the person using the treatment instrument 20, with the coupled sensor 40, has direct control at the operating site.

In one embodiment, the sensor 40 may be coupled with an external computer. The external computer may process the sensor data and determine the classification of tissue types and/or the action trigger by warning signals 60, 62, 64 and/or the generation of a deactivation signal 52 of the treatment device 20.

The processing unit may trigger further treatment devices, such as peripheral devices. A signal 70 may be generated on the basis of the measured values transmitted by the sensor 40. The signal may trigger a further medical instrument 72. The peripheral instrument 72 may be, for example, a suction device by which the removed tissue is removed directly from the operating area. This suction device could be activated simultaneously with the laser 22. The further peripheral instruments may be triggered. Each of the further peripheral instruments may be activated or deactivated according to their function and the tissue identified.

All kinds of medical instruments 20, irrespective of whether those instruments are provided for operations on human beings or on animals, may be used with the sensor 40.

The present embodiments are not restricted to the exemplary embodiment described above. Instead a plurality of variants and modifications are conceivable, which make use of the inventive thought and therefore also fall within the scope of protection.

1. A medical instrument for performing medical interventions on a body region, the medical instrument comprising:
at least one sensor for recording tissue parameters of a body region to be treated.

2. The medical instrument as claimed in claim 1, wherein the sensor is coupled with an evaluation unit.

3. The medical instrument as claimed in claim 2, wherein the evaluation unit comprises an output unit for outputting optical and/or acoustic signals.

4. The medical instrument as claimed in claim 1, wherein the evaluation unit comprises a signal processing unit for subdividing the examined body tissue into vulnerable body tissue and non-vulnerable body tissue.

5. The medical instrument as claimed in claim 1, wherein the evaluation unit includes a signal processing unit for identifying a classified tissue type.

6. The medical instrument as claimed in claim 1, wherein the evaluation unit is bidirectionally coupled.

7. The medical instrument as claimed in claim 1, wherein the evaluation unit is coupled with at least one further processing device.

8. The medical instrument as claimed in claim 1, wherein the sensor is an optical and/or an electrical sensor.

9. The medical instrument as claimed in claim 1, wherein the sensor comprises at least one transmitter and at least one receiver.

10. The medical instrument as claimed in claim 1 wherein the medical instrument is an endoscopic instrument, a scalpel, an HF scalpel, a medical robot, or a surgical laser.

11. The medical instrument as claimed in claim 5, wherein the classified tissue type includes malignant tissue.

12. A method for controlling a medical instrument during medical intervention, the method comprising:
identifying a tissue type with a sensor; and
controlling a function of the medical instrument as a function of the identified tissue type.

13. The method as claimed in claim 12, wherein the tissue type is healthy tissue or diseased tissue.

14. The method as claimed in claim 13, wherein controlling the function of the medical insturment includes stopping the fucntion of the medical instrument when healthy tissue is identified.

15. The method as claimed in claim 13, wherein controlling the function of the medical insturment includes starting the function of the medical instrument when diseased tissue is identified.
